(19) United States
(12) Patent Application Publication
(10) Pub. No.: US 2005/0226795 A1
(43) Pub. Date: Oct. 13, 2005

(54) Durable fill block for flow of fluids through a hinged lid

(75) Inventors: John A. Drummond, Hutto, TX (US); Robert R. Martinez, Austin, TX (US)

Correspondence Address: PAUL S MADAN;MADAN, MOSSMAN & SRIRAM, PC, 2603 AUGUSTA, SUITE 700, HOUSTON, TX, 77057-1130, US

(73) Assignee: Steriquip, Inc.

(21) Appl. No.: 11/098,337

(22) Filed: Apr. 4, 2005

Related U.S. Application Data
(60) Provisional application No. 60/559,470, filed on Apr. 5, 2004.

(51) Int. Cl. A61L 2/00

(52) U.S. Cl. 422/293; 422/296; 422/300; 206/438

(57) ABSTRACT

A fill block for injection of fluid through a hinged lid is improved by making the fluid pathway therethrough of a constant cross-sectional area, and by providing one or more rounded surfaces to reduce or eliminate impact and contact with surrounding structures when the lid is open and closed. Using a more durable and flexible material for the fill block and insert sheet increases the lifetime of both components and reduces or eliminates the tendency of these components to stress crack. Polycarbonate is one example of such durable and flexible material.

### CROSS-REFERENCE TO RELATED APPLICATION

This application claims the benefit of U.S. provisional application No. 60/559,470 filed Apr. 5, 2004.

### FIELD OF THE INVENTION

The present invention relates to methods and apparatus for allowing air to escape from a chamber having a hinged lid and allowing liquids introduced to the chamber to flow to a float switch indicating a full chamber, and more particularly relates, in one embodiment, to methods and apparatus for allowing air and liquids flow to and from a chamber having a hinged lid, where the flow of air and liquids is through the lid near the hinge.

### BACKGROUND OF THE INVENTION

Apparatus are known for having hinged lids that open to permit access to a chamber in the apparatus behind the lid. One example of such an apparatus is a sterilizing system where various objects such as immersible surgical and diagnostic devices, e.g. medical scopes and instruments are inserted to sterilize them. Generally, the lid is opened to insert the instrument and the lid is closed. One or more fluids may be injected, and the system may be under pressure during the sterilization. In some systems, fluid is allowed to escape from the sterilization chamber through an orifice in the hinged lid.

In some sterilizer system designs, there are blocks and structures with orifices and pathways therein near the hinge of the lid to channel the fluids from the chamber covered or sealed by the lid out of the system. Experience has shown that frequent and constant opening and closing of the lid not only stresses and damages these blocks and conduits, but has the potential of damaging the chamber interior, processing tray and/or structures of the system near the hinge. One structure that's often stressed to the cracking or breaking point is a polymer plastic insert sheet in the hinged lids of these systems. Some of these sterilizer systems have an inflatable seal that pushes the instrument tray into the insert at a great force. At other times, the operator may slam the lid which forces or hits the insert sheet into the contents of the chamber at more than the recommended and usual impact. Additionally, if an operator is not careful, the scopes and other instruments placed in the chamber for sterilizing may not be properly aligned or oriented and may extend into the space that the insert sheet normally occupies at the time the lid is closed resulting in damage both to the insert sheet and the instruments.

It would be desirable if the blocks and structures containing the pathways for injecting fluids through a hinged lid inhibited or prevented damage through repeated use and were more durable. Similarly, it would be useful if the insert sheet was more durable and flexible.

### SUMMARY OF THE INVENTION

Accordingly, it is an object of the present invention to provide a hinged lid assembly having greater durability.

It is another object of the present invention to provide a fill block connected to a hinged lid that is more efficient at permitting fluid to escape and flow through a system covered by the lid and that did not cause damage to surrounding structures that it touches or is in proximity to during opening and closing.

Another object of the invention is to provide a lid assembly that is more durable and less subject to damage or breaking.

In carrying out these and other objects of the invention, there is provided, in one form, a fill block for transport of fluid through a hinged lid. The fill block has a block body; a substantially flat contact face adapted to connect with an insert sheet; a fluid pathway having a first aperture on the flat contact face and a second aperture on a different portion of the block body than the flat contact face. The fill block also has at least one rounded surface to minimize impact with a structure when the insert sheet is closed or opened, where the insert sheet is connected to, integral with or the same thing as the hinged lid.

In another non-limiting embodiment of the invention, there is provided an assembly to permit transport of fluid through a hinged lid near a hinged edge that includes an insert sheet, where the insert sheet is connected to, integral with or the same thing as the hinged lid. The insert sheet includes a hinged insert edge along one side of the insert sheet, where the hinged edge is connected to, integral with or the same thing as a hinged insert edge of the hinged lid. The insert sheet also has at least one hinge on the hinged insert edge, where the hinge has an axis parallel to the hinged insert edge. The insert sheet also has an opening near the hinged insert edge. The fill block includes a block body; a substantially flat contact face adapted to connect with the insert sheet; a fluid pathway having a first aperture on the flat contact face and a second aperture on a different portion of the block body than the flat contact face. The first aperture of the fill block mates with, and is ultimately affixed around the opening of the insert sheet. The fill block also has at least one rounded surface to minimize impact with a structure when the lid is closed or opened, and alternatively when a seal inside the insert pushes a chamber processing tray up against the lid.

### BRIEF DESCRIPTION OF THE DRAWINGS

FIG. 1 is a schematic, three-quarters, perspective view of a sterilizing system with the hinged lid in open position showing one possible context of the invention;

FIG. 2 is an exploded, three-quarters, perspective view of one embodiment of the fill block of this invention and how it fits to an insert sheet and a backing plate;

FIG. 3A is an injection end view of one embodiment of the fill block of this invention;

FIG. 3B is a back view of the embodiment of the fill block of this invention shown in FIG. 3A;

FIG. 3C is a curved end view of the embodiment of the fill block of this invention shown in FIG. 3A;

FIG. 3D is a side view of the embodiment of the fill block of this invention shown in FIG. 3A;

FIG. 3E is a front view of the embodiment of the fill block of this invention shown in FIG. 3A; and

FIG. 3F is an end view of an alternate, prior art fill block showing a beveled edge.

### DETAILED DESCRIPTION OF THE INVENTION

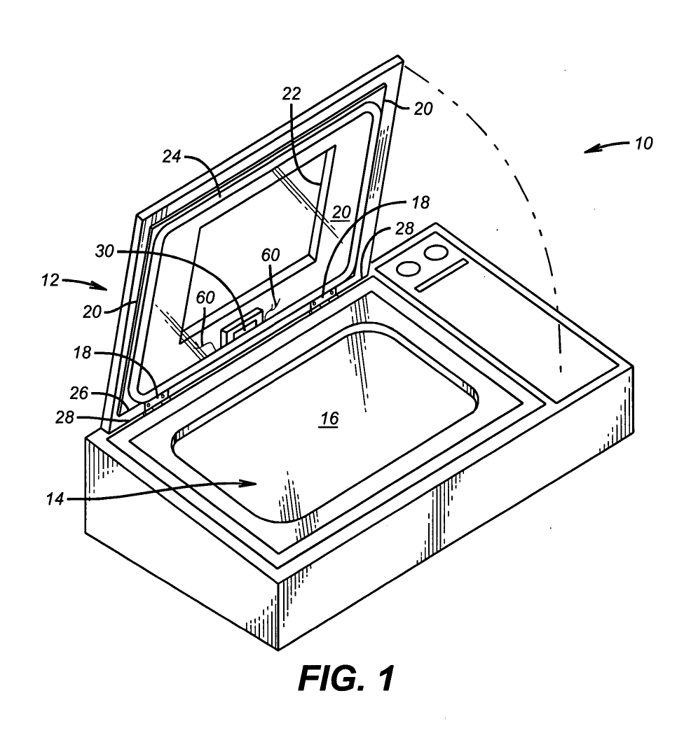

Shown in FIG. 1 is one non-limiting embodiment of a sterilizing system or device 10 having a hinged lid 12 and an interior chamber 14. The interior chamber 14 may include, but is not necessarily limited to, a processing tray 16 shown in this particular embodiment, but also a structure integral with device 10, a gasket, a spacer, a cover, a handle, a frame, and other like structures.

In the particular embodiment shown in FIG. 1, the hinged lid 12 is in the open position bearing on hinges 18. The number of hinges 18 is not important so far as hinged lid 12 pivots or swings on at least one hinge 18. Lid 12 has an insert sheet 20 within it. Insert sheet 20 may be connected to, integral with or the same thing as hinged lid 12 in various non-limiting embodiments. Insert sheet 20 may be transparent and lid 12 may have a window 22 therein to permit viewing of anything within chamber 14, as in the FIG. 1 embodiment. Lid 12 may also have a gasket 24 for forming a seal with the chamber 14 or other interior of system or device 10, such as in an embodiment where the chamber is under pressure. The gasket 24 may be glued to the insert and may be made of EPDM (ethylene-propylene diene monomer) or silicone rubber or other suitable material. In one non-limiting embodiment, the lid 12 may be metal, and in one non-restrictive alternative may be aluminum, stainless steel, or other suitable metal. In one non-limiting embodiment, the hinge 18 is connected to the metal part of the lid 12, and hinge 18 is not connected to the insert 20. However, in other embodiments lid 12 and insert 20 may be one or otherwise integral with each other.

The insert sheet 20 of lid 12 has a insert edge 26 adjacent to, connected to, integral with or the same thing as a hinged edge 28 of lid 12. Insert edge 26 is provided with an opening 30 to permit fluids and air to be passed or drained from chamber 14 through lid 12 or alternatively injected, introduced or placed into chamber 14 through opening 30 in lid 12. In another non-limiting embodiment, air relief is the only fluid entering or leaving opening 30. Vacuum relief may be defined herein as the chamber 14 draining out water or other fluids through a port (not shown) in the chamber 14 bottom.

In one non-limiting embodiment of the invention, and simply to give a sense of scale, the system or device 10 seen in FIG. 1 may have a width of about 32 inches, a depth of about 24 inches, a height of about 13 inches, and a weight of about 150 pounds.

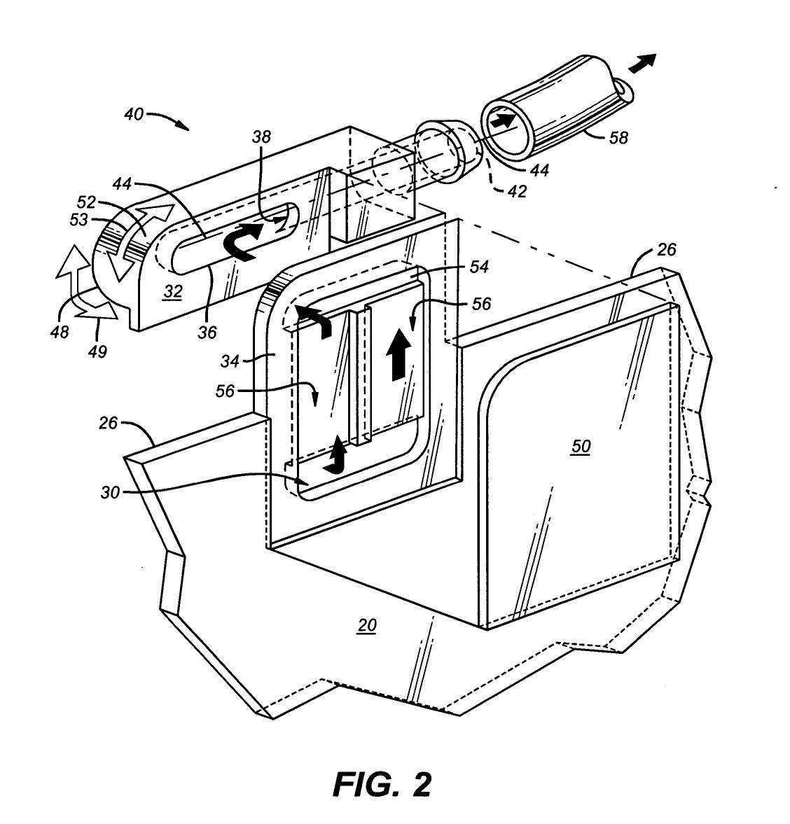

Shown in FIG. 2 is a detailed, exploded, three-quarters, perspective view of one embodiment of the fill block 40 of this invention and how it fits to an insert sheet 20 and a backing plate 50. As compared with FIG. 1, the insert sheet 20 in FIG. 2 is upside-down and viewed from the other side. Opening 30 is shown in insert sheet 20 where the fluid and air would flow from the chamber 14 (not shown) in a direction out from the Figure toward the viewer. In contrast, in FIG. 1, the fluid would go through opening 30 into the Figure, away from the viewer. It will be appreciated that the direction of flow shown in FIG. 2 (dark arrows) is consistent with an embodiment where chamber 14 is filled with a fluid (liquid or air) from another source or opening not shown and drained through opening 30. In an alternate version of the invention, chamber 14 would be filled by a fluid from hose 58 flowing opposite or against the dark arrows and the chamber 14 would be drained by a different port (not shown).

In one non-restrictive embodiment, liquid flow from hose 58 (in the direction of the dark arrows of FIG. 2) would go to a float switch or other valve or detector (not shown) to indicate that chamber 14 was full and to shut off liquid flow thereto. As part of a sterilizing cycle, in one embodiment of the invention, air (generally speaking, a fluid) would flow back through into the chamber 14 in the reverse direction from that indicated by the dark arrows in FIG. 2.

In one non-limiting embodiment of the invention, fill block 40, insert sheet 20, and backing plate 50 would be transparent, and indeed, this would be a commercial embodiment of the invention, as shown. However, it may be understood that one or more of the elements being discussed or elements of the invention may be opaque or translucent, rather than transparent, and the invention should not be limited by any of these optical properties.

FIG. 2 shows fill block 40 in detail, including the block body and a substantially flat contact face 32 adapted to connect to, mate with or be adhered to insert sheet 20, in this embodiment specifically by extension or tab 34 which includes opening 54 that mates with first aperture 36 on the contact face 32 of fill block 40. By “substantially flat” is meant of sufficient planarity to fit flush to another similar substantially flat surface, such as insert 20, and to be affixed, adhered or glued thereto. Fill block 40 also has a fluid pathway 38 extending from a second aperture 42 through block 40 to first aperture 36. In the particular embodiment shown in FIG. 2, the fluid pathway 38 has a main axis 44, where the main axis is perpendicular to first aperture 36, and the main axis 44 is oriented parallel to the axes of the hinges 18 that open and close insert sheet 20 and hinged lid 12, shown more particularly in FIG. 1. This orientation places second aperture 42 at a right angle or 90° to first aperture 36. While this orientation is not necessarily required, it is expected that it may find advantages in this invention, such as being more readily consistent and/or congruent with the operation of opening and closing of hinged insert sheet 20.

It will be appreciated that embodiments of the invention can be envisioned where insert sheet 20 does not have an extension or tab 34, but where fill block 40 fits flush with a straight or linear insert edge 26.

In one non-limiting embodiment of the invention, fluid pathway 38 has a uniform cross-section. However, fluid pathway 38 does not necessarily have to have the same shape to have the same cross-sectional area in this embodiment. A fluid pathway 38 having a uniform cross-section does not block, impede or inhibit fluid flow, and thus does not inhibit fluid flow due to such obstruction, narrowing or blocking, which may be undesirable in some cases. In another non-limiting embodiment of the invention, the opening 54 in the insert sheet 20 is at least as large as the cross-sectional area of fluid pathway 38 for the same reason. In one non-restrictive version of the invention, and to give a sense of scale in one embodiment of the invention, this uniform cross-sectional area would be about the area of a circular opening ⅜-inch (about 1 cm) in diameter or about 0.1 in² (0.7 cm²).

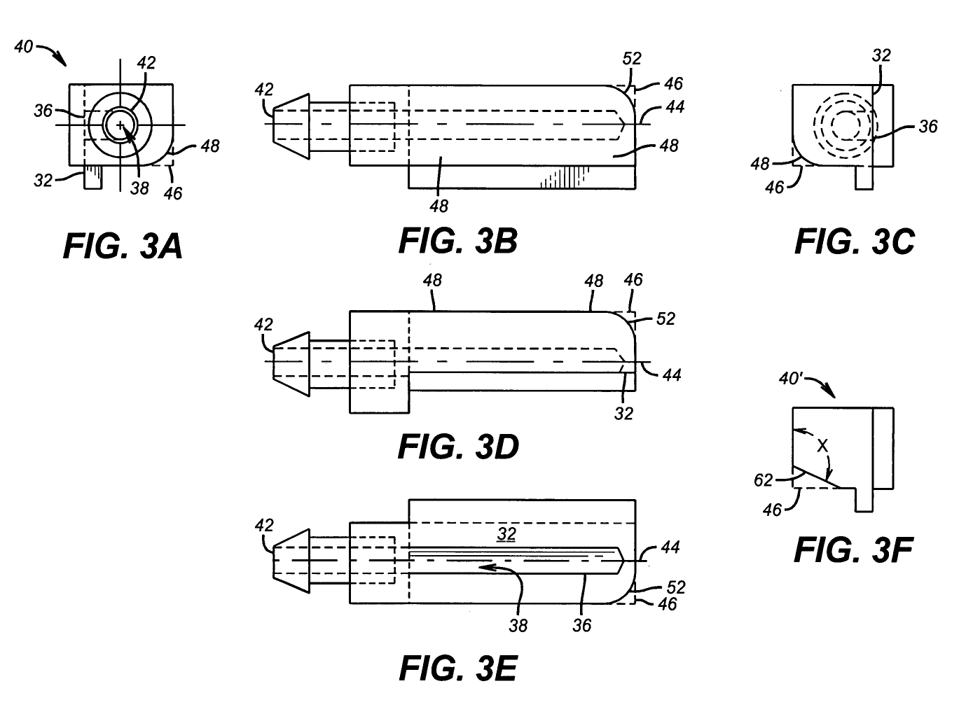

Fill block 40 also has at least one rounded surface to minimize impact with a structure when the insert sheet 20 is open or closed. This structure is typically device 10 itself, a processing tray 16, a gasket, a spacer, a cover, a handle, a frame or some other structure in proximity to block 40 when it is open or closed. With reference to FIGS. 3A-3E, typically, conventional fill blocks have sharp corners or edges 46 as shown in phantom lines in all FIGS. 3A through 3E. Shown in FIG. 3F is an alternate, prior fill block 40′ where sharp edge 46 is replaced by beveled edge 62. The designers of this prior fill block realized the difficulties caused by sharp edge 46, and beveled the edge to an angle x of about 120°. However, even this beveling did not sufficiently minimize impact with a structure as described above, and cracks 60 such as shown in FIG. 1 would still result.

In one non-limiting embodiment of the invention, a first curved or rounded surface is surface or rounded edge 48, which is a rounded surface generally parallel to the axes of hinges 18 to avoid or limit contact with other structures as insert sheet 20 is open and closed. Curved edge 48 is curved in the direction of arrow 49 in FIG. 2 and edge 48 extends along the back side of block 40 as seen in FIG. 2.

Another rounded surface is rounded corner 52, which also serves the function of limiting or preventing contact with other structures during the opening and closing of insert sheet 20. Rounded corner 52 is curved in the direction of arrow 53 as seen on the left side of block 40 seen in FIG. 2. To give some sense of scale, in one non-limiting embodiment of the invention fill block 40 may be about 2.5 inches long, not including the nozzle second aperture 42 shown (which may optionally be a separate piece), about 1 inch tall across the flat contact face, and about 0.8 inch wide across the top at its widest point.

With further reference to FIG. 2, it may be seen that fill block 40 is designed to connect to or mate with insert sheet 20 so that first aperture 36 on block 40 mates with opening 54, in this embodiment on extension or tab 34. Tab 34 may be provided with at least one passageway 56 from opening 54 to opening 30 to provide fluid communication therewith. In the particular embodiment shown in FIG. 2, two passageways 56 of generally flat, rectilinear, planar shape are formed, where one major wall thereof is provided by backing plate 50. In one non-limiting embodiment of the invention, the total cross-sectional area of passageways 56 and of opening 30 are the same as that of fluid pathway 38, again to avoid inhibiting or blocking fluid flow. Block 40, insert sheet 20 and backing plate 50 may be made of plastics or polymers as will be further described below, and may be adhered, glued, connected and/or assembled as shown in FIG. 2. FIG. 2 also shows flexible hose 58 that may be removably attached to second aperture 42 on fill block 40.

Thus, the fluid pathway to drain or exit fluid from chamber 14 of device or system 20 (shown in FIG. 1, but not FIG. 2) is as shown by the dark arrows in FIG. 2, generally out opening 30 in chamber 14 and via passageways 56 of sheet 20 through opening 54 of insert sheet 20 and first aperture 36 of fill block 40 through fluid pathway 38, second aperture 42, and out hose 58. As mentioned, the invention is not restricted by the flow direction of the particular embodiment of a system 20.

Commercial lids, fill blocks and backing plates are made of PLEXIGLASS acrylic polymers. In a hospital environment, sterilization systems used for the terminal sterilization of surgical scopes and instruments operate at temperatures of from about 35 to 40° C. minimum up to about 50 to 55° C. maximum or more over a cycle time of about 30 to 45 minutes. The water pressure entering such systems is often controlled at about 40 to about 50 psig. During a sterilization cycle, a flow of about 4 gallons per minute (gpm) may enter the system at an approximate pressure of about 40 psig.

A sterilization cycle is initiated when the lid is closed. Typically, secure lid closure is monitored throughout the cycle. An air compressor is enabled and controlled by pressure switches pressurizing an inflatable seal or gasket (such as 24, although such seal or gasket could be alternatively or additionally in the base of the unit or system as well), which locks the lid closed and seals the processing or sterilizing chamber. In an alternate embodiment, the seal is not inflatable. The switch maintains the air manifold at 35 to 45 psig, in one non-limiting embodiment, turning the compressor on and off as necessary.

A function of some of these sterilizing systems is to allow a processing tray—such as a removable tray used to route a sterilant or biocidal agent (e.g. peracetic acid, hydrogen peroxide or other peroxide, and the like) in and around a surgical scope, instruments, or the like—to be pushed up against the lid gasket in order to seal the chamber. This may be accomplished by the inflatable seal pushing the tray upward when inflated in another non-limiting embodiment.

It may thus be readily seen that the materials of the fill block, insert sheet and backing plate are subjected to a variety of severe conditions including, but not necessarily limited to, elevated temperatures, elevated pressures, a sterilizing and/or cleansing environment and chemicals (e.g. peracetic acid, peroxyacetic acid, hydrogen peroxide, and the like), as well as mechanical stresses due to repeated and frequent opening of the lid and assembly that cause the various parts and elements of the assembly to hit and impact one another, in addition to aging. It has been found that acrylic polymers over time and the conditions mentioned may form cracks 60 in the locations shown on insert sheet 12 in FIG. 1 as well as other locations. Stresses on insert sheet 12 also occur because of impact with or contact with an inflatable seal in the body of the sterilizing system 10, and by the insert sheet 12 being slammed closed with too much force, and/or by impact with a scope or other instrument that is not properly placed in processing tray 16 and extending outward into space normally occupied by insert sheet 12.

It has been further discovered that fill block 40, as well as insert sheet 20 and/or backing plate 50 are advantageously made of polycarbonate polymers and copolymers. Polycarbonates have the additional advantages of being transparent, and readily assembled using industrial and engineering adhesives or welded using a solvent methyl chloride, methylene chloride, and the like. In one non-limiting embodiment, a suitable adhesive is E6000 adhesive. Other polymers may be used in accordance with the method of this invention as long as they meet the requirements and fulfill the purposes and goals outlined herein. Thus, one advantage of the invention is the implementing of more durable and stress resistant polycarbonate or other material instead of less flexible and durable acrylic polymers which have a tendency to crack under normal operating procedures. Polycarbonates have a number of advantages including, but not necessarily limited to, being shatter-resistant, being harder than acrylic polymers, and being able to withstand temperatures of at least 250° F. Polycarbonates are of the group of thermoset polymers which does not melt or reflow and cannot be remolded; polycarbonates are used where strength and heat resistance is required. Polycarbonate insert sheets are also more flexible than acrylic polymers when the lid 12 is closed and chamber 14 is placed under pressure and thus further resist cracking.

In another non-restrictive embodiment of the invention, polycarbonate insert sheets 20 may be adhered to metal hinged lids 12, such as those made of aluminum, e.g., by means of an adhesive. Suitable adhesives may include, but are not necessarily limited to, silicone RTV (room temperature vulcanizing) adhesives. A specific, non-restrictive example of a suitable silicone RTV adhesive is Dow Corning® 832 multi-surface adhesive sealant. In some cases, it may be necessary to improve the adhesion of insert 20 on an aluminum lid 12 by abrading or roughening the surface of the insert 20 and/or by using a primer. Suitable primers include, but are not necessarily limited to, mixtures of inorganic and organic compounds, in non-restrictive examples one or more of toluene, alkylene glycol alkyl ethers such as propylene glycol methyl ether, alkyl glycol acetates such as butyl glycol acetate, alkoxyalcohols such as 2-methoxypropanol, and silanes such as methoxysilane and/or aminoethylaminopropyltrimethoxysilane. In one non-limiting embodiment of the invention, a specific, suitable primer may be Dow Corning® 1205 prime coat.

Thus, the fill block 40 of the present invention is of a better configuration and more suitably designed to cause less problems upon repeated movement of the lid 12, and is of a more durable and sturdy material, as compared with prior structures.

In the foregoing specification, the invention has been described with reference to specific embodiments thereof, and is expected to be effective in providing a structure and configuration that prolongs the life of the fill block and hinged lid. However, it will be evident that various modifications and changes can be made to the inventive apparatus without departing from the broader spirit or scope of the invention as set forth in the appended claims. Accordingly, the specification is to be regarded in an illustrative rather than a restrictive sense. For example, specific combinations of designs and materials falling within the claimed parameters, but not specifically identified or tried in a particular apparatus or device, or under specific conditions, are anticipated to be within the scope of this invention.

1. A fill block for transport of fluid through a hinged lid comprising:
a block body;
a substantially flat contact face adapted to connect with an insert sheet;
a fluid pathway having a first aperture on the flat contact face and a second aperture on a different portion of the block body than the flat contact face;
at least one rounded surface to minimize impact with a structure when the insert sheet is closed or opened, where the insert sheet is connected to, integral with or the same thing as the hinged lid.

2. The fill block of claim 1 where the fluid pathway has a uniform cross-sectional area from aperture to aperture.

3. The fill block of claim 1 where the fill block comprises polycarbonate.

4. The fill block of claim 1 where the fluid pathway has a main axis, where the main axis is perpendicular to the first aperture, and where the main axis is parallel to the axis of at least one hinge that opens and closes the insert sheet.

5. The fill block of claim 4 where the at least one rounded edge is generally parallel to the axis of hinges and proximal to the insert sheet.

6. The fill block of claim 1 where the fill block further comprises at least one rounded corner to minimize impact with a structure when the insert sheet is closed or opened.

7. A fill block for transport of fluid through a hinged lid comprising:
a block body;
a substantially flat contact face adapted to connect with an insert sheet;
a fluid pathway having a first aperture on the flat contact face and a second aperture on a different portion of the block body than the flat contact face, where the fluid pathway has a main axis, where the main axis is perpendicular to the first aperture, and where the main axis is parallel to the axis of hinges that open and close the insert sheet;
at least one rounded edge generally parallel to the axis of hinges and proximal to the insert sheet, the rounded edge to minimize impact with a structure when the insert sheet is closed or opened, where the insert sheet is connected to, integral with or the same thing as the hinged lid.

8. The fill block of claim 7 where the fluid pathway has a uniform cross-sectional area from aperture to aperture.

9. The fill block of claim 7 where the fill block comprises polycarbonate.

10. An assembly to permit transport of fluid through a hinged lid near a hinged edge thereof comprising:
an insert sheet where the insert sheet is connected to, integral with or the same thing as the hinged lid, the insert sheet comprising:
a hinged insert edge along one side of the insert sheet, where the hinged insert edge is connected to, integral with or the same thing as a hinged edge of the hinged lid;
at least one hinge on the hinged insert edge, where the hinge has an axis parallel to the hinged insert edge and
an opening near the hinged insert edge; and
fill block comprising:
a block body;
a substantially flat contact face adapted to connect with the insert sheet;
a fluid pathway having a first aperture on the flat contact face and a second aperture on a different portion of the block body than the flat contact face, where the first aperture mates with the opening of the insert sheet; and
at least one rounded surface to minimize impact with a structure when the insert sheet is closed or opened.

11. The assembly of claim 10 where the fluid pathway of the fill block has a uniform cross-sectional area from aperture to aperture.

12. The assembly of claim 11 where the opening in the insert sheet is at least as large as the uniform cross-sectional area.

13. The assembly of claim 10 where the fluid pathway of the fill block has a main axis, where the main axis is perpendicular to the first aperture, and where the main axis is parallel to the axis of the hinge that opens and closes the insert sheet.

14. The assembly of claim 10 where rounded surface of the fill block is at least one rounded edge generally parallel to the axis of hinges and proximal to the insert sheet.

15. The assembly of claim 10 where the fill block further comprises at least one rounded corner to minimize impact with a structure when the insert sheet is closed or opened.

16. The assembly of claim 10 where the fill block and the insert sheet comprise polycarbonate.

17. The assembly of claim 10 further comprising a device where the hinged lid is subjected to pressure and the structure is selected from the group consisting of the device itself, a tray within the device, a gasket, a spacer, a cover, a handle, a frame, and combinations thereof.

18. The assembly of claim 10 where the opening near the hinged insert edge is a first opening, and where the assembly further comprises a second opening in the insert sheet and at least one passageway providing fluid communication between the first and second opening.

19. The assembly of claim 18 where the fluid pathway, first aperture, first opening, passageway and second opening all have the same uniform cross-sectional area.

20. The assembly of claim 10 where the hinged edge is the hinged edge of the insert sheet and where the insert sheet is affixed to a separate hinged lid, where the hinged lid is metal.
